(19) United States
(12) Patent Application Publication
(10) Pub. No.: US 2004/0210955 A1
(43) Pub. Date: Oct. 21, 2004

(54) Preparation of spermatozoa for ICSI-mediated transgenesis and methods of using the same

(76) Inventors: Hidenori Akutsu, Baltimore, MD (US); Tomoharu Osada, Kyoto (JP); Ryuzo Yanagimachi, Honolulu, HI (US)

Correspondence Address: Leslie Gladstone Restaino, Esq., Brown Raysman Millstein Felder & Steiner LLP, 163 Madison Avenue, P.O. Box 1989, Morristown, NJ, 07962-1989, US

(21) Appl. No.: 10/280,898

(22) Filed: Oct. 25, 2002

Related U.S. Application Data
(60) Provisional application No. 60/348,171, filed on Oct. 26, 2001.

(51) Int. Cl. C12N 15/85

(52) U.S. Cl. 800/21; 435/455

(57) ABSTRACT

The present invention provides methods of preparing spermatozoa suitable for use in ICSI-mediated transgenesis, wherein the methods include the suspension of spermatozoa in a buffered medium comprising an ion-chelating agent. In a preferred embodiment of the invention, the method of preparing spermatozoa for ICSI-mediated transgenesis further comprises treatment of membrane-disrupted or demembranated spermatozoa with a disulfide reducing agent. Also provided are spermatozoa suitable for use in ICSI-mediated transgenesis, wherein the exogenous nucleic acid to be co-inserted in an unfertilized oocyte via ICSI is closely associated with the membrane-disrupted or demembranated spermatozoon. Finally, a method for obtaining a transgenic embryo is disclosed, comprising the steps of coinserting a membrane-disrupted or demembranated spermatozoon of the present invention and an exogenous nucleic acid into an unfertilized oocyte to form a transgenic fertilized oocyte, and thereafter allowing the transgenic fertilized oocyte to develop into a transgenic embryo. If so desired, the transgenic embryo may be transplanted into a surrogate mother and allowed to develop into a live transgenic offspring.

### RELATED APPLICATION

This application claims the benefit of U.S. Provisional Application No. 60/348,171, the contents of which are incorporated herein in their entirety.

### BACKGROUND OF THE INVENTION

Perhaps few areas of applied genetics have generated as much interest, or hold as much promise, as the field of transgenics. Potential applications of transgenic animals include improved milk and meat production, engineered resistance to common diseases of commercially valuable livestock, the production of whole-animal expression cloning systems, or “bioreactors”, to generate large amounts of biopharmaceutical and industrial gene products, and the development of transgenic animals to yield “non-immunogenic” cells and tissues for xenotransplantation into humans.

The use of transgenic animals has also greatly advanced the understanding of functional genomics, particularly in the areas of neurobiology, immunology, developmental biology and cancer biology. By way of example, transgenic animals may be used to confirm and validate cell culture investigations of cis-acting regulatory elements. Or a transgene may be inserted into a target gene to produce a null mutation phenotype in the transgenic animal, with the further insertion of a mutant copy of the endogenous target gene used to create an animal model of human disease. Further, transgenic copies of an endogenous gene may be used for over-expression and ectopic expression studies. These are but a few of the avenues for research and application.

Transgenesis involves the transfer of exogenous DNA into totipotent (capable of differentiating into all of the cells of an adult organism) or pluripotent (capable of differentiating into a large number, though not all, of the cells of an adult organism) embryonic cells, followed, generally, by integration of the transferred DNA into the host chromosomes. Pronuclear microinjection is the most commonly used method to create a transgenic animal (J. W. Gordon, et al., Genetic Transformation of mouse embryos by microinjection of purified DNA. Proc. Natl. Acad. Sci. U.S.A. 77, 7380, 1980; J. W. Gordon and F. H. Ruddle, Integration and stable germ line transmission of genes injected into mouse pronuclei. Science 214, 1244, 1981; R. D. Palmiter and R. L. Brinster, Germ-line transformation of mice. Annu. Rev. Genet. 20, 465, 1986; and J. W. Gordon, Transgenic animals. Int. Rev. Cytol. 115, 171, 1989). In this method, a transgene, often coupled to a tissue specific promoter, is injected through a microinjection pipette into a newly fertilized oocyte recovered from a superovulated female. A surviving embryo is cultured, and then implanted into the oviduct of a pseudopregnant surrogate female, where it is allowed to develop into a mature animal. The microinjected transgene randomly integrates into the DNA chromosome, usually at a single site, thereby allowing transmission to subsequent generations in a mendelian fashion.

Generation of transgenic zygotes via pronuclear microinjection has been straightforward in the mouse, but this has not been true for species exemplified by the large commercial breeds. (Perry, A., et al., Mammalian Transgenesis by Intracytoplasmic Sperm Injection. Science, 284: 1180-1183, 1999). One reason is that zygotes are difficult substrates for pronuclear microinjection when their lipid richness renders them opaque, as in cattle and pigs, whereas mouse zygotes are translucent. (Perry, A., et al., supra). Furthermore, the use of a very fine microinjection pipette (typically with an aperture of 1 micrometer in diameter, or less) generates shearing forces that would limit the handling of large nucleic acid constructs, such as BACs and YACs.

Mammalian transgenesis may also be mediated through intracytoplasmic sperm injection (Perry, A., et al., supra), where a membrane-disrupted or demembranated spermatozoon or sperm head, together with exogenous DNA, is coinjected into a metaphase II oocyte. The resulting transgenic embryo may then be transplanted into a surrogate mother and allowed to develop into a live transgenic offspring. ICSI-mediated transgenesis offers a number of advantages over pronuclear microinjection. For example, the use of pipettes with a 100-fold larger tip aperture (˜78 μm² for a pronuclear microinjection tip of diameter of 1 μm, compared with ˜78 μm² for an ICSI tip of diameter 10 μm) facilitates the handling of large megabase and sub-megabase constructs such as bacterial, yeast or mammalian artificial chromosomes. The association of exogenous nucleic acid with the demembranated or membrane-disrupted spermatozoa or sperm heads offers additional stabilization and protection of these constructs.

Further, zygotes are difficult substrates for pronuclear microinjection when their lipid richness renders them opaque, as is the case with cattle and pigs. Accordingly, the use of unfertilized metaphase II oocytes as a substrate in ICSI represents a major facilitatory simplification over other methods (such as pronuclear microinjection) that require zygotes.

There is a need, however, to increase the efficiency of ICSI-mediated transgenesis. Reported efficiencies of ICSI-mediated transgenesis are currently on the order of 1.8%, expressed as a function of the proportion of transgenic offspring developed from 100 gene-injected oocytes (Perry, A., et al., supra), which is generally comparable to transgenesis mediated by pronuclear microinjection. Processes for demembranating or membrane-disrupting spermatozoa prior to co-injection into an unfertilized oocyte (including mechanical disruption, freeze-thawing, freeze-drying and detergent extraction) may release endogenous nucleases (Maione, B., et al., Activation of endogenous nucleases in mature sperm cells upon interaction with exogenous DNA. DNA Cell. Biol., 16:1087-1097, 1997), thereby causing chromosomal or sub-chromosomal damage that potentially limits the efficiency of ICSI-mediated transgenesis. Further, the stability of the sperm nucleus in a number of mammalian species, partly a function of the association of sperm DNA with protamines, may inhibit the decondensation of the sperm nucleus and formation of the male pronucleus, both of which are required for proper embryonic development. (Rho, et al., Sperm and oocyte treatments to improve the formation of male and female pronuclei and subsequent development following intracytoplasmic sperm injection into bovine oocytes. Biol. Reprod., 59(4): 918-924, 1998).

Chelating agents have been used widely in research directed to spermatozoa fertility and potency. In particular, chelating agents have been studied both as fertility inhibitors, e.g., as spermicide additives (Yu et al., Int. J. Androl., 10(6):741-46, 1987), and as fertility promoters, e.g., as enhancers of chromatin decondensation. For example, Rodriguez et al. have demonstrated that the use of chelating agents to bind metal ions that are present in semen results in enhanced chromatin decondensation in ram sperm (Rodriguez et al, Int. J. Androl., 8(2):147-58, 1985). Furthermore, U.S. Pat. No. 5,773,217, issued to L. J. Wangh, discloses use of chromatin-decondensation-enhancing chelating agents to pretreat permeabilized sperm cells prior to sperm-nucleus activation. Nevertheless, chelating agents are also known to exhibit unwanted aneuploidogenic properties that can lead to chromosomal aberrations (Russo et al., Environ. Mol. Mutagen., 19(2):125-31, 1992).

Chelating agents with fertility-promoting properties have also been added to spermatozoa suspension media in non-frozen storage applications. WO 02/24872 describes a nuclear-extraction buffer containing chromatin-decondensation-enhancing chelating agents for washing and storing sperm samples prior to analysis or interaction with other cells or media. However, the addition of chelating agents to an ambient-temperature storage solution, while maintaining the oocyte-penetrating ability of sperm, promotes a high rate of intracellular metabolic activity that may lead to chromatin damage and chromosomal abnormalities (Vishwanath et al., Reprod. Fertil. Dev., 9(3):321-31, 1999). Other studies have utilized chelating agents as an ancillary additive to a physiological medium using trehalose as a cryoprotectant, where the chelating agents prevent cation competition with the cyroprotectant for membrane-binding sites (Aisen et al., Theriogenology, 53(5):1053-61, 2000).

To date, however, no known studies have investigated the use of chelating agents to increase the efficiency of ICSI-mediated transgenesis.

Various studies have also examined the use of disulfide reducers, such as dithiothrietol (DTT) thioredoxin, as a means of improving ICSI-mediated fertilization. By way of example, Rho et al. have determined that the efficiency of bovine intracytoplasmic sperm injection can be improved by sperm pretreatment with DTT. (Rho, et al., supra). Embryo transfer of ICSI fertilized zygotes resulted in the pregnancy in 6 of 16 recipients, but none of these pregnancies carried to term.

Suttner et al. compared seven different protocols, including pretreatment of sperm with DTT, on the activation and fertilization rates of bovine oocytes after ICSI and on their subsequent development in in vitro conditions. (Intracytoplasmic sperm injection in bovine: effects of oocyte activation, sperm pretreatment and injection technique. Theriogenology, 54(6): 935-948, 2000). It was found that pretreatment of sperm with DTT could improve the success of bovine ICSI, though no in vivo studies were undertaken.

Pronuclear formation and development were examined for the effects of sperm pretreatment with DTT and oocyte activation with ethanol at ICSI. (Asada, et al., An attempt at intracytoplasmic sperm injection of frozen-thawed minke whale (Balaenoptera bonaerensis) oocytes. Zygote, 9(4):299-307, 2001). 2- and 4-cell cleaved oocytes were observed after injection of sperm pretreated with DTT, although no in vivo studies were performed. (Asada, et al., supra).

The use of DTT to promote decondensation of the sperm nucleus in vitro was also reported by Chung, et al., in “Activation of bovine oocytes following intracytoplasmic sperm injection (ICSI).” (Theriogenology, 53(6):1273-1284, 2000). The study, however, was directed primarily to the investigation of various methods of oocyte activation, and there was no independent determination of whether the pretreatment of sperm with DTT improved ICSI-mediated fertilization rates.

While the foregoing references disclose the use of DTT in ICSI-mediated fertilization, none disclose the use of DTT to increase the efficiency of ICSI-mediated transgenesis. Further, none of Rho, et al. (Biol. Reprod., 59(4): 918-924, 1998), Suttner, et al. (Theriogenology, 54(6): 935-948, 2000), Asada, et al. (Zygote, 9(4):299-307, 2001), or Chung, et al. (Theriogenology, 53(6):1273-1284, 2000), disclose any live births resulting from ICSI.

Accordingly, there exists a need for protocols that increase the efficiency of ICSI-mediated transgenesis.

### SUMMARY OF THE INVENTION

In accordance with the present invention, there is provided a method of preparing a spermatozoon suitable for use in ICSI-mediated transgenesis. In one embodiment of the present invention, the method comprises the steps of suspending the spermatozoon in a buffered medium, wherein the buffered medium comprises an ion-chelating agent, treating the spermatozoon to obtain a membrane-disrupted or demembranated spermatozoon, and incubating the membrane-disrupted or demembranated spermatozoon with an exogenous nucleic acid for a period of time. In a preferred embodiment, the ion-chelating agent is a divalent ion chelating agent, such as a calcium chelating agent. In the most preferred embodiment, the calcium chelating agent is ethylene glycol-O,O′-bis-[2-amino-ethyl]-N,N,N′,N′,-tetraacetic acid (EGTA).

In an additional and preferred embodiment, a method of preparing a spermatozoon suitable for use in ICSI-mediated transgenesis is disclosed, where the method comprises the steps of: suspending the spermatozoon in a buffered medium, wherein the buffered medium comprises an ion-chelating agent; treating the spermatozoon to obtain a membrane-disrupted or demembranated spermatozoon; incubating the membrane-disrupted or demembranated spermatozoon with a disulfide reducing agent for a period of time; and incubating the membrane-disrupted or demembranated spermatozoon with an exogenous nucleic acid for a period of time. The disulfide reducing agent is preferably dithiothrietol (DTT), also referred to in the art as Cleland's reagent, 1-4-Dimercapto-2,3-butanediol, DL-dithiothrietol, and RAC-dithiothreitol.

The present invention further provides a membrane-disrupted or demembranated spermatozoon suitable for ICSI-mediated transgenesis, wherein the exogenous nucleic acid to be co-inserted in an unfertilized oocyte via ICSI is closely associated with the membrane-disrupted or demembranated spermatozoon. In addition, a method for obtaining a transgenic embryo is disclosed, comprising the steps of coinserting the membrane-disrupted or demembranated spermatozoon of the present invention and the exogenous nucleic acid into an unfertilized oocyte to form a transgenic fertilized oocyte, and thereafter allowing the transgenic fertilized oocyte to develop into a transgenic embryo. If so desired, the transgenic embryo may be transplanted into a surrogate mother and allowed to develop into a live transgenic offspring.

Additional aspects of the present invention will be apparent in view of the description that follows.

### DETAILED DESCRIPTION OF THE INVENTION

Standard protocols for ICSI-mediated transgenesis (as described fully in U.S. Pat. No. 6,376,743, entitled “Mammalian transgenesis by intracytoplasmic sperm injection”, and Perry, et al., Mammalian Transgenesis by Intracytoplasmic Sperm Injection. Science, 284: 1180-1183, 1999, the contents of each of which are expressly incorporated herein by reference) involve a number of general steps, including, to wit: obtaining spermatozoa from the desired donor animal; subjecting the spermatozoa to various processes (as described below and in U.S. Pat. No. 6,376,743) to yield either membrane-disrupted or demembranated spermatozoa or sperm heads; incubating a exogenous nucleic acid with the membrane-disrupted or demembranated spermatozoa or sperm heads; co-inserting the exogenous nucleic acid and a membrane-disrupted or demembranated spermatozoon or sperm head into an unfertilized oocyte to form a transgenic transgenic fertilized oocyte; and allowing the transgenic fertilized oocyte to develop into a transgenic embryo. If desired, the transgenic embryo may be transplanted into a suitable surrogate mother and allowed to develop into a transgenic live offspring.

While comparable to the prevailing method of pro-nuclear microinjection-mediated transgenesis, the efficiency of ICSI-mediated transgenesis (defined as the proportion of transgenic offspring developed per 100 intracytoplasmic sperm/exogenous nucleic acid injected oocytes) is low, approximately on the order of 1.8% (Perry, et al., supra). The inventors have shown herein that the efficiency of ICSI-mediated transgenesis may be significantly increased by a special preparation of the spermatozoa or sperm heads, wherein the special preparation comprises the steps of suspending the spermatozoa or sperm heads in a medium comprising an ion-chelating agent prior to sperm disruption or demembranation, and, in a preferred embodiment, pretreating the spermatozoa or sperm heads with a disulfide-reducing agent prior to exposure of the spermatozoa or sperm heads to the exogenous nucleic acid. In an exemplary protocol described herein below, the efficiency of ICSI-mediated transgenesis was raised to 6.8%, a substantial improvement over standard protocols previously known in the art.

Accordingly, the present invention provides a method of preparing a spermatozoon suitable for use in ICSI-mediated transgenesis, wherein the method comprises the steps of: suspending the spermatozoon in a buffered medium, wherein the buffered medium comprises an ion-chelating agent; treating the spermatozoon to obtain a membrane-disrupted or demembranated spermatozoon; and incubating the membrane-disrupted or demembranated spermatozoon with an exogenous nucleic acid for a period of time.

In a preferred embodiment of the present invention, a method of preparing a spermatozoon suitable for ICSI-mediated transgenesis is disclosed, wherein the method comprises the steps of: suspending the spermatozoon in a buffered medium, wherein the buffered medium comprises an ion-chelating agent; treating the spermatozoon to obtain a membrane-disrupted or demembranated spermatozoon; incubating the membrane-disrupted or demembranated spermatozoon with a disulfide reducing agent for a period of time; and incubating the membrane-disrupted or demembranated spermatozoon with an exogenous nucleic acid for a period of time.

In a preferred embodiment of the invention, the spermatozoon is a complete, physiologically mature spermatozoon, or even more preferably, is a sperm head thereof, where a “sperm head” is defined as a sperm fragment containing all of the head components, including the nucleus. It is understood that “sperm head” may be substituted herein for spermatozoon as a vehicle for ICSI-mediated transgenesis wherever appropriate.

The nuclear DNA of physiologically mature spermatozoa, or of the sperm heads thereof, is associated with basic proteins called protamines. In mammals, protamines are extensively cross-linked by disulfide bonds, which have the effect of stabilizing the sperm nuclei and rendering them very resistant to physical and chemical disruption. While the cross-linking by disulfide bonds may render sperm nuclei more resistant to physical and chemical disruption, in some species such stability interferes with nuclear decondensation and pronuclear formation following ICSI, both of which are necessary for proper development of the zygote. Such interference may be reduced by pre-treating the spermatozoa with a disulfide reducer prior to ICSI, as described in further detail below.

Cross-linking of nuclear protamines occurs mainly during transit of the spermatozoa through the epididymis. Thus, mammalian spermatozoa within the epididymis and in ejaculate (semen) are generally physiologically more mature than those within the testis, and are preferred in the methods of the present invention—at least in mammals.

Mature spermatozoa from invertebrates and vertebrates are collected by methods known to those skilled in the art. For example, mature spermatozoa of rodents, such as mouse, golden (Syrian) hamster, guinea pig, and the like, may be collected from caudae epididymes; contrastingly, in other species, such as humans, rabbits, pigs, horses, bulls, goats, fowl, and the like, mature spermatozoa may be isolated from freshly-ejaculated semen of fertile males. Spermatozoa of fish (e.g., swordtail, Xiphophorus helleri) and invertebrates, such as sea urchins (Tripneustes gratilla), may be collected from the testes of mature males.

By way of example, mouse spermatozoa may be obtained from a cauda epididymis by the following method. A cauda epididymis is removed from a mature male mouse (approximately 8 weeks after birth or older). The blood and adipose time are removed from the surface of the cauda epididymis. The cauda epididymis is then compressed to release a dense mass of spermatozoa. A drop (about 2 μl) of sperm mass is placed in the bottom of centrifuge tubes containing 1.5 ml polypropylene, and overlain with 0.5 ml of warm buffered medium comprising an ion-chelating agent (e.g., CZB medium, phosphate-buffered saline (PBS), EGTA medium (as defined herein), or isotonic saline). After about 10-20 min at 37° C., motile spermatozoa may be collected from the supernatant.

Additionally, by way of example, spermatozoa may be obtained from semen by the following method. Freshly-ejaculated human semen is allowed to liquefy for about 30 min at room temperature (about 25° C.). The semen is then diluted with about 10 ml of saline, and filtered through about two layers of tissue paper to remove debris. The filtrate may then be centrifuged at 400×g for about 10 min, and the sedimented spermatozoa resuspended in a buffered solution or medium comprising an ion-chelating agent, at a desired concentration, for subsequent treatment (e.g., freeze-thawing, freeze-drying, mechanical disruption or detergent extraction) to obtain membrane-disrupted or demembranated spermatozoa.

Spermatozoa may be obtained from a testis, for example, by the following method. An excised testis is placed in an erythrocyte-lysing buffer (e.g., 155 mM NH₄Cl, 10 mM KHCO₃, 2 mM EDTA; pH 7.2-7.4), minced using a pair of fine scissors, and filtered through about two layers of tissue paper to remove debris. The filtrate is then centrifuged (e.g., 700×g, for 5 min), and the pellet is resuspended in a buffered medium or solution comprising an ion-chelating agent, at a desired concentration, in preparation for subsequent treatment (e.g., freeze-thawing, freeze-drying, mechanical disruption or detergent extraction) to obtain membrane-disrupted or demembranated spermatozoa.

Alternatively, in another embodiment of the invention, a spermatozoon of the present invention may be a pre-spermatozoal cell (i.e., a male germ cell before it has been transformed into motile a spermatozoon), such as a round spermatid cell. The use of round spermatids would provide advantages in cases of testicular failure resulting in maturation arrest, among other situations.

According to the method of sperm preparation disclosed herein, the spermatozoa are suspended in a buffered medium, wherein the buffered medium optimally comprises an ion-chelating agent. As defined herein, a “buffered medium” is a solution or other liquid material that is prepared for the growth, maintenance or storage of biological material, and comprises a chemical capable of maintaining the pH of the solution or medium by absorbing hydrogen ions (which would make it more acidic) or hydroxyl ions (which would make it more basic). For example, in the present invention, use of a buffer may maintain the pH of the spermatozoa suspension in a range between 7.2 and 8.6. Preferably, the pH of the spermatozoa suspension ranges between 7.4 and 8.2.

The buffered medium of the present invention may be a medium used to suspend or store spermatozoa (e.g., a sperm-suspension medium). While spermatozoa can be suspended in a variety of media, a physiological suspension or storage medium is frequently used. A physiological medium is one that maintains the tissues of an organism in a viable state. Such a medium contains specific concentrations of substances that are vital for normal tissue function (e.g., bicarbonate and phosphate ions, calcium, chloride, glucose, magnesium, oxygen, potassium, and sodium), and also has an appropriate osmotic pressure. One example of a physiological medium is Ringer's solution, which is an aqueous solution containing sodium chloride, potassium chloride, and calcium chloride, and has an osmotic pressure the same as that of blood serum. Other examples of buffered media for use in the present invention include, without limitation, CZB medium, Earle's Balanced Salt Solution (EBSS) (designed for use in a 5% CO₂ atmosphere), Hank's Balanced Salt Solution (HBSS) (for use in an air atmosphere in closed containers), Hepes buffer, isotonic saline, phosphate-buffered saline (PBS), Tris-HCl buffer, Tyrode's solution (a salt solution which is a modified Locke solution, comprising NaCl, KCl, CaCl₂.6H₂O, MgCl₂.6H₂O, NaHCO₃, NaH₂PO₄, glucose, and distilled water), and any others described herein. Preferably, the buffered medium is Tris-HCl buffer (e.g., 10 mM Tris-HCl buffer).

In the method of the present invention, the buffered medium comprises an ion-chelating agent, so that the spermatozoon is suspended in a buffered medium comprising an ion-chelating agent prior to treatment of the spermatozoon by freeze-thawing, freeze-drying, detergent extraction or mechanical disruption to obtain a demembranated or membrane-disrupted spermatozoon. Preferably, the ion-chelating agent is a divalent-cation chelating agent. Ion-chelating agents are chemical compounds that form complexes with metal ions by serving as multidentate ligands. In particular, an ion-chelating agent is an organic chemical that bonds with free metal ions, and removes them from solutions. A single chelating agent may form several bonds with a single metal ion. Chelator-ion complexes are quite stable in solution, and are common vehicles for transporting metal ions in biological systems. Examples of ion-chelating agents for use in the present invention include, without limitation, EDTA (ethylene diamine tetra-acetic acid), EGTA (ethylene glycol-bis (β-aminoethylether)-N,N,N′,N′-tetra-acetic acid), and EGTA-AM (the acetoxymethyl ester of EGTA). The concentration of the ion-chelating agent is preferably between about 0.1 mM and about 200 mM, more preferably between about 1 mM and about 100 mM, and especially between about 40 mM and about 60 mM.

Without being bound by theory, it is likely that structural chromosome aberrations are caused by the release of endogenous nucleases from plasma-membrane-damaged spermatozoa following sperm-head isolation, freeze-drying, or freezing without cryoprotection. Maione et al. (DNA Cell. Biol., 16:1087-97, 1997) reported the existence of Ca²⁺-dependent endogenous nucleases in mouse spermatozoa. Accordingly, in a preferred embodiment of the present invention, an ion-chelating agent binds ions such as magnesium and calcium. In an even more preferred embodiment of the present invention, the ion-chelating agent is the calcium-chelating agent, EGTA, and the concentration of EGTA is 50 mM. Preferred concentrations of EGTA for use in the present invention are described in further detail as follows.

The following exemplary solutions, comprising 10 mM Tris-HCl buffer and varying concentrations of NaCl and EGTA, may be used in the composition of the present invention: (1) 20 mM NaCl and 50 mM EGTA; (2) 50 mM NaCl and 50 mM EGTA; (3) 50 mM NaCl and 10 mM EGTA; and (4) 80 mM NaCl and 50 mM EGTA. These solutions may be prepared, for example, from stock solutions of 5 M NaCl, 0.5 M EGTA (pH 8.0, adjusted with NaOH), and 1 M Tris-HCl (pH 7.4) previously made up and diluted with ultrapure water (Millipore Systems). Chemicals may be obtained from Sigma Chemical Co. (St. Louis, Mo.).

Following suspension of the spermatozoa in a suitable buffered medium, one which preferably comprises an ion-chelating agent as described above, the spermatozoa are treated to obtain membrane-disrupted or demembranated spermatozoa. Methods for disrupting or demembranating spermatozoa are described in detail in U.S. Pat. No. 6,376,743, entitled “Mammalian transgenesis by intracytoplasmic sperm injection”, the contents of which are expressly incorporated herein.

Membrane-disrupted spermatozoa have sperm membranes that have been rendered permeable by a number of methods (Rho, et al., supra), including immobilizing sperm and damaging the sperm membrane by freezing and thawing before injection; rehydrating freeze-dried spermatozoa (Wakayama and Yanigimachi, Development of normal mice from oocytes injected with freeze-dried spermatozoa. Nature Biotechnology, 16:639, 1998); and crushing the sperm with the micropipette used for injection (Lacham-Kaplan and Trounson, Micromanipulation assisted fertilization: comparison of different techniques. In: Tesarik J (ed), Male Factor in Human Infertility. Rome, Italy: Ares-Serono Symposia Publications 1994).

Freeze-thawed spermatozoa may be prepared according to the methods described in T. Wakayama, et al., (Production of normal offspring from mouse oocytes injected with spermatozoa cryopreserved with or without cryoprotection. J. Reprod. Fert. 112: 11, 1998) and S. Kuretake, et al, (Biology of Reproduction 55: 789, 1996).

In the exemplary method for freezing mouse epididymal spermatozoa, a drop of sperm mass from the cauda epididymis was placed at the bottom of a 1.5 ml polypropylene microcentrifuge tube (Fisher Scientific, Pittsburgh, Pa.), and overlaid with 200 μL of EGTA medium (50 mM EGTA, 50 mM NaCl and 10 mM Tris-HCl, pH 7.4-8.2). The vial was tightly capped and plunged into liquid nitrogen (−196° C.) for 10 seconds, and thawed by hand warming. The thawed sperm suspension is now ready for incubation with a disulfide reducing agent, or, alternatively, with an exogenous nucleic acid, prior to use in intracytoplasmic sperm injection (ICSI), as described below. Although one method of obtaining freeze-thawed sperm has been described herein for mouse epididymal spermatozoa, one of ordinary skill in the art may utilize other methods of freeze-thawing, or adapt the method to spermatozoa from other vertebrates and invertebrates without undue experimentation.

Freeze-drying spermatozoa also results in disruption of the plasma membrane, as assessed by viability staining techniques that are capable of distinguishing between plasma membrane-intact (live) and plasma membrane-damaged (dead) cells (e.g., by Live/Dead FertiLight, Molecular Probes, Oreg.). Such freeze-dried membrane-disrupted spermatozoa are considered “dead” in the conventional sense. Freeze-dried spermatozoa may be prepared according to the methods described in T. Wakayama and R. Yanagimachi, Nature Biotechnology 16, 639, (1998) and in copending U.S. patent application Ser. No. 09/177,391, filed Oct. 23, 1998, the contents of each of which are incorporated herein in their entirety. In particular, general methods that may be used for freeze-drying spermatozoa from vertebrates and invertebrates are disclosed.

In an exemplary method for freezing mouse epididymal spermatozoa, the sperm concentration in CZB, DMEM or EGTA medium is about 3 to 10×10⁶ per ml. An aliquot (100 μl) of the sperm suspension is put in a 2 ml ampoule (Wheaton Scientific, Millville, N.J., Catalogue No. 651506), and is thereafter plunged directly into liquid nitrogen. Ten minutes later, ampoules are placed in a pre-cooled (−50° C.) freeze-flask attached to a freeze-dry system (Model 10-020, VirTis Co., Gardner, N.Y.). The inlet pressure is approximately 1 milliTorr. About 12 hours later, the flask is removed from the system after it has been filled with argon supplied by way of a gas-drying jar (Fisher Scientific, Pittsburgh, Pa. Catalogue No. 09-204). Each ampoule is connected to a vacuum pump and frame-sealed after more than greater than 99% of the gas is pumped out of it. Ampoules are individually wrapped with aluminum foil and stored in the dark at room temperature (about 25° C.) or at 4° C. for up to a year prior to use.

Freeze-dried spermatozoa must be rehydrated prior to incubation with a disulfide reducer and/or incubation with an exogenous nucleic acid. The freeze-dried-sperm is preferably rehydrated by adding pure water, the volume of which is the same as the original volume of the sperm suspension before freeze-drying. Once rehydrated, any physiological salt solution, such as 0.9% saline or CZB medium, may be used for dilution; the dilution volume is not critical. The concentration of spermatozoa in the final rehydration medium should be sufficient to facilitate the retrieval of individual sperm or individual sperm heads for purposes of sperm injection into oocytes, as described below.

Although one method of obtaining rehydrated freeze-dried sperm has been described herein for mouse epididymal spermatozoa, one of ordinary skill in the art may utilize other methods of freeze-drying, or adapt the method to spermatozoa from other vertebrates and invertebrates, without undue experimentation, as taught in U.S. patent application Ser. No. 09/177,391.

Finally, the membranes of fresh spermatozoa as described above may be disrupted by mechanical means, such as by dislocation of sperm heads from tails in the microinjection pipette by the application of a single pulse from a piezo-electrically actuated microinjection unit, as described further below. As used herein, the term “fresh” spermatozoa refers to such membrane-disrupted spermatozoa for microinjection into unfertilized oocytes, and these are distinguished from, and represent a difference from, “live” spermatozoa used as vehicles for DNA delivery in previous reports of IVF.

Demembranated spermatozoa or sperm heads are detergent-extracted spermatozoa or sperm heads that lack all membranes, including the plasma membrane and inner and outer acrosomal membranes, but retain the nucleus and perinuclear material. For example, sperm heads may be demembranated by treatment with Triton X-100, with or without SDS (sodium dodecyl sulfate). Triton X-100 is a well-known, non-ionic surfactant that is widely used for removal of membrane components under non-denaturing conditions. SDS is an anionic detergent used to solubilize various proteins, including membrane proteins. In the mouse, sperm heads demembranated by Triton X-100 have been shown to be capable of activating oocytes, leading to normal embryonic development.

In the preferred method of the present invention, following the treatment of the spermatozoa to obtain membrane-disrupted or demembranated spermatozoa, and prior to incubation of the membrane-disrupted or demembranated spermatozoa with an exogenous nucleic acid, the membrane-disrupted or demembranated spermatozoa are incubated with a disulfide reducing agent for a period of time.

As used herein, a “disulfide reducing agent” is an agent that specifically reduces disulfide bonds. In mammals, the nuclear structures of spermatozoa are associated with basic proteins called protamines, which become extensively cross-linked by disulfide bonds during maturation. This cross-linking has effect of stabilizing the sperm nuclei and rendering them very resistant to physical and chemical disruption. While the cross-linking by disulfide bonds may render sperm nuclei more resistant to physical and chemical disruption, in some species such stability interferes with nuclear decondensation and pronuclear formation following ICSI, both of which are necessary for proper development of the zygote. Accordingly, pre-treating the spermatozoa with a disulfide reducer prior to ICSI-mediated transgenesis, as described in further detail below, may act to decondense the sperm DNA and aid in the formation of the male pronucleus following ICSI. Without being bound by theory, it is further speculated that the higher rates of efficiency observed in ICSI-mediated transgenesis using the method of the present invention are due to the increased opportunity for association between the exogenous nucleic acid and the decondensed nuclear material (DNA) of the membrane-disrupted or demembranated spermatozoa.

The disulfide reducer of the present invention may be any one of dithiothreitol (DTT), tris-(2-carboxyethyl) phosphine (TCEP), tris-(2-cyanoethyl) phosphine (TCP), thioredoxin (TRX) or glutathione (GSH), and in a preferred embodiment of the invention is DTT. DTT is also referred to in the art as Cleland's reagent, 1-4-Dimercapto-2,3-butanediol, DL-dithiothrietol, and RAC-dithiothreitol. The disulfide reducer of the present invention may be suspended in any suitable buffer medium (e.g., buffered saline solution) and added to the membrane-disrupted or demembranated spermatozoa for incubation in varying concentrations. In one embodiment of the invention, the concentration of disulfide reducer during incubation with the membrane-disrupted or demembranated spermatozoa is between 0.1 mM and about 50 mM, is more preferably between 1 mM and about 5 mM, and, where the disulfide reducer is DTT, is most preferably at 1 mM. The optimal temperature of incubation should be between 0° C. and 4° C.

The length of incubation of the membrane-disrupted or demembranated spermatozoa with the disulfide reducing agent should be sufficient to result in a partial or complete nuclear decondensation of the membrane-disrupted or demembranated spermatozoon, so as to allow for association between the exogenous nucleic acid and the nuclear material of the demembranated or membrane-disrupted spermatozoa. Degrees of nuclear decondensation may be confirmed through phase contrast microscopy. In one embodiment of the present method, the incubation time period is about 5 minutes to 1 hour, is more preferably about 10 minutes to 50 minutes, and is even more preferably about 20 minutes to 40 minutes. In the most preferred embodiment, the incubation time period is about 30 minutes, the disulfide reducing agent is DTT in 1 mM concentration, and the temperature of incubation is between 0° C. and 4° C.

The method of the present invention comprises the additional step of incubating the membrane-disrupted or demembranated spermatozoa with an exogenous nucleic acid for a period of time. Following incubation with the exogenous nucleic acid, the spermatozoa are ready to be used in ICSI-mediated transgenesis, as described further below.

As used herein, an exogenous nucleic acid is comprised of genetic material that is not indigenous to (not normally resident in) the zygote (i.e., not indigenous to the unfertilized oocyte or to the membrane-disrupted or demembranated spermatozoon or sperm head) before transformation or is not normally present in more than one copy. However, it is contemplated that an exogenous nucleic acid may also include a further copy of an indigenous gene or genetic sequence that is introduced for purposes of over-expression or co-suppression of a target gene product, or may include a copy of indigenous genetic material that has been modified by the addition, deletion, substitution and/or alteration of one or more nucleotides, including non-naturally occurring nucleotides.

The exogenous nucleic acid may comprise DNA from any origin including, but not limited to, plants, bacteria, viruses, bacteriophage, plasmids, phasmids, plastids, avians, fish, amphibians, reptiles, invertebrates (such as, by way of example, sea urchins, lobsters, shellfish or abalones), mammals (such as, by way of example, primates, ovines, bovines, porcines, ursines, felines, canines, equines or rodents), and synthetic DNA constructs. The exogenous nucleic acid may comprise cDNA or, in a preferred embodiment, genomic DNA, where the gene or genes of interest are operably linked to, and under the control of, regulatory DNA sequences that permit expression of the gene or genes in the transgenic embryo or animal. The DNA may code, together with the appropriate tissue specific or non-tissue specific expression cassette (as described in further detail below), for the expression of any number of products, including, but not limited to, a cytotoxin, an immunomodulatory protein, a tumor antigen, a growth factor, a hormone, a vaccine antigen, an antisense RNA molecule, a ribozyme, a non-coding small RNA molecule, or a signal transduction enzyme. The DNA may be in circular or linear form and may be single-stranded or double-stranded. The DNA may be inserted into the host cell DNA in a sense or anti-sense configuration and in single-stranded or double-stranded form. All or part of the DNA inserted into the host cell may be integrated into the genome of the host. The exogenous nucleic acid may comprise more than one transgene; either on the same or separate nucleic acid strands.

Examples of non tissue-specific promoters that might be used in the exogenous nucleic acid of the present invention include, without limitation: the SV40 early or late promoter (Southern, P. J. & Berg, P., Transformation of mammalian cells to antibiotic resistance with a bacterial gene under control of the SV40 early region promoter. J. Mol. Appl. Genet. 1: 327-341, 1982); the cytomegalovirus immediate early (CMV-IE) promoter (U.S. Pat. No. 4,168,062); the cytoplasmic β-actin promoter; and the adenovirus major late promoter. Examples of tissue specific promoters that might be used in exogenous nucleic acid of the present invention are enumerated in U.S. Pat. No. 6,277,621, entitled “Artificial chromosome constructs containing foreign nucleic acid sequences”, and include, without limitation: the desmin promoter, which is specific for muscle cells; the enolase promoter, which is specific for neurons; the β-globin promoter, which is specific for erythroid cells; the tau-globin promoter, which is also specific for erytliroid cells; the growth hormone promoter, which is specific for pituitary cells; the insulin promoter, which is specific for pancreatic beta cells; the glial fibrillary acidic protein promoter, which is specific for astrocytes; the tyrosine hydroxylase promoter, which is specific for catecholaminergic neurons; the amyloid precursor protein promoter, which is specific for neurons; the dopamine β-hydroxylase promoter, which is specific for noradrenergic and adrenergic neurons; the tryptophan hydroxylase promoter, which is specific for serotonin/pineal gland cells; the choline acetyltransferase promoter, which is specific for cholinergic neurons; the aromatic L-amino acid decarboxylase (AADC) promoter, which is specific for catccholaminergic/5-HT/D-type cells; the proenkephalin promoter, which is specific for neuronal/spermatogenic epididymal cells; the reg (pancreatic stone protein) promoter, which is specific for colon and rectal tumors, and pancreas and kidney cells; and the parathyroid hormone-related peptide (PTHrP) promoter, which is specific for liver and cecum tumors, and neurilemoma, kidney, pancreas, and adrenal cells.

The successful delivery of the DNA into a cell may be preliminarily evaluated by the expression of a “reporter” gene. A reporter gene is a component of the DNA used for transformation and may be the same as or different than the transgene conferring another desired property. The property conferred on the transformed cell or tissue by the reporter gene is usually easily detectable by histochemical or fluorescence assays. There are a number of commonly used in vitro reporter genes for quantifying transfection efficiencies, and numerous plasmids and cloning vectors containing reporter transgenes are available from commercial sources, known to those skilled in the art, such as Sratagene, Inc., LaJolla, Calif., and Clontech Laboratories, Inc., Palo Alto, Calif. Exemplary reporter genes for use in the present invention include, but are not limited to, secreted alkaline phosphatase [SEAP; β-galactosidase (β-gal); firefly luciferase, and chloramphenicol acetyltransferase (CAT)]. In vivo reporter assays, such as in situ β-gal staining, in situ β-glucuronidase [GUS] and in situ luciferase assays are also available for detecting gene transfer in either fixed cells or tissue sections. These procedures allow visualization of transfected cells following staining with enzymatic substrates or antibodies. Among these procedures, in situ β-gal staining following expression of the Escherichia coli LacZ gene is a widely used method because of its simplicity and sensitivity. In this procedure, reaction of β-gal with the X-gal substrate produces a rich blue color that can be easily visualized under a light microscope and, there, provides a direct assessment of transfection efficiency.

The green fluorescent protein (GFP) from the jellyfish Aequorea Victoria has become an important reporter for monitoring gene expression and protein localization in a variety of cells and organisms (R. Y. Tsien, The green fluorescent protein. Annu. Rev. Biochem. 67: 509, 1998; G. Zhang, et al., An enhanced green fluorescent protein allows sensitive detection of gene transfer in mammalian cells. Biochemical and Biophysical Research Communications 227: 707-711, 1996; T. Takada, et al., Selective production of transgenic mice using green fluorescent protein as a marker. Nature Biotechnology 15: 458-460, 1997). Because GFP does not require any substrate for detection, it can be a suitable marker for the selection of transgenic embryos. GFP expressed in eukaryotic cells yields green fluorescence when cells are excited by UV or blue light. The chromophore in GFP is intrinsic to the primary structure of the protein, and fluorescence from GFP does not require additional cofactors, substrates, or additional gene products. GFP fluorescence is stable, species-independent, and can be monitored noninvasively using techniques of fluorescence microscopy, flow cytometry, and macroscopic imaging. To increase the fluorescent intensity of GFP when excited by blue light, an enhanced GFP (EGFP) variant has been constructed (pEGFP-Cl available from Clontech Laboratories) that contains the immediate early promoter of human CMV and SV40 polyadenylation signals to drive expression of the EGFP gene in mammalian cells.

Selection and/or synthetic construction of plasmids and other cloning vectors containing specific genes are well known in the art. Synthetic constructs of chimeric plasmids contain the gene or genes of interest and frequently comprise promoter and/or leader sequences obtained from diverse sources to facilitate insertion into the host genome. Although prokaryotic cloning vector sequences have no apparent effect on the integration frequency of microinjected genes, it has been noted that they can severely inhibit the expression of eukaryotic genes introduced into a germ line of a mammal, such as a mouse (see B. Hogan, et al., in Manipulating the Mouse Embryo, Section E, Second Ed., Cold Spring Harbor Laboratory Press, p. 22, 1994). Therefore, it may be advisable to remove substantially all vector sequences from a cloned gene before introducing it into the germ line of a mammal, such as a mouse, if optimal expression of the gene is desired. Vector sequences may be removed by employing restriction enzymes, according to the restriction sites present on the vector, by methods known to those skilled in the art, to produce fragments containing the desired gene, promoters, enhancers, and the like.

However, conventional vectors, such as plasmids, phages, cosmids, or viral vectors, may be of limited use when introducing very large DNA fragments into an unfertilized oocyte to form a transgenic embryo. Accordingly, as used herein, an exogenous nucleic acid may also be a very large DNA fragment greater than 30 kilobases in length. The ultimate size of the very large DNA fragment is limited only by the carrying capacity of the chosen vector. The choice of vector will be apparent to one of ordinary skill in the art, and will depend on a number of factors, including, but not limited to: the size of the DNA fragment; the taxonomic classification of the host animal; and whether the very large DNA fragment is to be integrated into the host genome or is to maintained stably and independently as an extrachromosomal element. Exemplary vectors include bacterial artificial chromosomes, or BACs (Shizuya, H., et al., Cloning and stable maintenance of 300-kilobase-pair fragments of human DNA in Eschericia coli using an F-factor-based vector. Proc. Natl. Acad. Sci. USA 89:8794-8797, 1992); P1 based artificial chromosomes or PACs (Ioannou, P. A., et al., A new bacteriophage P1-derived vector for the propagation of large human DNA fragments. Nat. Genet. 6:84-89, 1994); yeast artificial chromosomes or YACs (Burke, D. T., et al., Cloning of large segments of exogenous DNA into yeast by means of artificial chromosome vectors. Science 236:806-812, 1987; Larin, Z., et al., Generation of a large insert YAC library. Methods Mol. Biol. 54: 1-11, 1996; Olson, et al., U.S. Pat. No. 4,889,806, entitled “Large DNA cloning system based on yeast artificial chromosomes”; see also, Loring, et al., U.S. Pat. No. 5,981,175, entitled “Methods for producing recombinant mammalian cells harboring a yeast artificial chromosome”); mammalian artificial chromosomes or MACs (Vos, J. M. H., The simplicity of complex MACs. Nature Biotech. 15:1257-1259, 1997; Harrington, et al., U.S. Pat. No. 6,348,353, entitled “Artificial Mammalian Chromosome”; Scheffler, Immo, U.S. Pat. No. 5,721,118, entitled “Mammalian artificial chromosomes and methods of using same”), which include, by way of example, satellite DNA based artificial chromosomes or SATACs (Hadlaczky, Gyula, Satellite DNA-based artificial chromosomes for use in gene therapy. Curr. Opin. Mol. Thera. 3(2): 125-132, 2001; Hadlaczky, et al., U.S. Pat. No. 6,025,155, entitled “Artificial chromosomes, uses thereof and methods for preparing artificial chromosomes”); minichromosomes (Farr, et al., Generation of human X-derived minichromosome using telomere associated chromosome fragmentation. EMBO J. 14:5444-5454, 1995; Heller, et al., Minichromosomes derived from human Y chromosome by telomere directed chromosome breakage. Proc Natl Acad Sci USA 93:7125-7130, 1996); and human artificial chromosomes, or HACs (Kuroiwa, Y. et al., Manipulation of human minichromosomes to carry greater than megabase-sized chromosome inserts. Nat. Biotechnol. 18:1086-1090, 2000).

In a preferred embodiment of the invention, the membrane-disrupted or demembranated spermatozoa as prepared above are mixed with the exogenous nucleic acid following a period of incubation of the membrane-disrupted or demembranated spermatozoa with a disulfide reducing agent, preferably DTT. In a typical mixing procedure, 1 μL of a DNA solution containing the exogenous nucleic acid (about 2.5 ng/μL) is mixed with 9 μL of a suspension containing about 2 to 5×10⁵ spermatozoa in a physiological medium, such as CZB or NIM, and mixed by pipetting to give a final DNA fragment concentration of around 7 ng/μL. The mixture is incubated at room temperature (about 25° C.) or on ice for about 30 seconds to about 5 minutes, typically about 45 seconds to about 3 minutes, more typically about one to about 3 minutes, preferably about one minute. In any event, the incubating time period with the exogenous nucleic acid should be sufficient to form a close association between the exogenous nucleic acid and the membrane-disrupted or demembranated spermatozoa. The concentration of sperm and exogenous nucleic acid may be varied, as well as the incubation times and temperatures, depending on the size of the DNA, or the size of the sperm, and the like, as known to those skilled in the art.

The demembranated and membrane-disrupted spermatozoa prepared according to the methods of the present invention are closely associated with the exogenous nucleic acid, and as such are extremely suitable for use in high efficiency ICSI-mediated transgenesis. As used herein, “closely associated” means that the sperm submembrane compartments interact and form non-covalent bonds with the exogenous nucleic acid in a manner that promotes transgenesis. Accordingly, the present invention provides for the spermatozoa prepared according to the methods disclosed herein, namely: suspension of the spermatozoa in a buffered medium, which comprises a ion chelating agent such as EGTA; treatment of the spermatozoa (by mechanical disruption, freeze-thawing, freeze-drying and rehydration, or detergent extraction) to obtain membrane-disrupted or demembranated spermatozoa; preferably, incubation of the membrane-disrupted or demembranated spermatozoa with a disulfide reducing agent for a period of time; and incubation of the membrane-disrupted or demembranated spermatozoa with an exogenous nucleic acid for a period of time.

Spermatozoa prepared according to the methods disclosed herein are suitable for injection into an unfertilized oocyte via ICSI, using standard ICSI protocols as described in depth in U.S. Pat. No. 6,376,743, entitled “Mammalian transgenesis by intracytoplasmic sperm injection”, the contents of which with regard to ICSI protocol are expressly incorporated herein by reference. Prior to co-injection of the demembranated or membrane-disrupted spermatozoa and exogenous nucleic acid into unfertilized oocytes, the sperm-nucleic acid suspension may be mixed with a concentration of polyvinyl pyrrolidone (PVP; mw=360,000) (ICN Pharmaceuticals, Inc., Costa Mesa, Calif.) in Hepes-buffered CZB medium. PVP acts to prevent the spermatozoa from sticking to the inner surface of the micropipette and reduces their motility for easier handling during ICSI. Further, the mixing step acts to dilute any concentration of ion-chelating agent, such as EGTA, that may be present in the sperm-nucleic acid suspension, as the introduction of EGTA into the oocyte cytoplasm may inhibit oocyte activation.

Accordingly, in an additional method of the present invention, the demembranated or membrane-disrupted spermatozoa which have been incubated with the exogenous nucleic acid are washed and/or diluted prior to use in ICSI-mediated transgenesis, such as by the addition of a buffered medium, addition of a solution comprising a concentration of PVP or similar substance, or by standard washing techniques known to those of ordinary skill in the art, e.g., via centrifugation and resuspension in a suitable buffered medium.

A method for obtaining a transgenic embryo is disclosed, comprising the steps of coinserting the spermatozoon prepared by any method of the present invention, together with its closely associated exogenous nucleic acid, into an unfertilized oocyte to form a transgenic fertilized oocyte, and thereafter allowing the transgenic fertilized oocyte to develop into a transgenic embryo. If desired, the transgenic embryo may be transplanted into a surrogate mother and allowed to develop into a live offspring.

The method of the present invention may be used to produce transgenic embryos or live offspring of mammals, such as primates, ovines, bovines, porcines, ursines, felines, canines, equines and rodents. The method may also be used to produce transgenic invertebrates such as, but not limited to sea urchins, lobster, abalone or shell fish. The method may also be used to produce transgenic fish, amphibians, reptiles and birds. It has been discovered herein that live transgenic offspring (founder animals) produced by the process of the invention are themselves capable of producing transgenic offspring, showing stable integration of the exogenous nucleic acid into the founder genome as well as the fertility of the founders.

The present invention is described in the following Examples, which are set forth to aid in the understanding of the invention, and should not be construed to limit in any way the scope of the invention as defined in the claims which follow thereafter.

### EXAMPLES

The following example illustrates the methods of the present invention, and the uses thereof in methods for developing live offspring from oocytes injected with reconstituted freeze-dried spermatozoa. In particular, the examples illustrate the development of normal mice from mouse oocytes injected with the heads (nuclei) of reconstituted freeze-dried mouse spermatozoa. The sperm-suspension medium prior to freeze-drying contained a buffer and an ion-chelating agent, EGTA, as described below.

### Example 1

### Animals

Gametes were obtained from B6D2F1 (C57BL/6X DBA/2) female and B6D2F1 male mice, aged 8-12 weeks. Random-bred CD-I albino females, 8-12 weeks old, which had been mated with vasectomized CD-1 males, were used as recipients for morulae/blastocyst transfer on the third day of pseudopregnancy. All animals were maintained according to the guidelines prepared by the Committee on Care and Use of Laboratory Animals of the Institute Resources National Research Council (DHEW Publication No. [NiH] 80-23, revised in 1985).

### Example 2

### Reagents and Media

All chemicals were obtained from Sigma Chemical Co. (St. Louis, Mo.), unless otherwise stated.

A solution comprising 10 mM Tris-HCl buffer, 50 mM NaCl and 50 mM EGTA (EGTA medium) was used for suspending spermatozoa for freezing. The EGTA medium was prepared from stock solutions of 5 M NaCl, 0.5 M EGTA (pH 8.0, adjusted with NaOH), and 1 M Tris-HCl (pH 7.4) previously made up and diluted with ultrapure water (Millipore Systems, Burlington, Mass.). The pH of the final EGTA medium was 7.4-8.2.

Harvested oocytes were kept in CZB medium (Chatot, et al., An improved culture medium supports development of random-bred 1-cell mouse embryos in vitro, J. Reprod. Fert., 86, 679-688 1989) prior to coinjection of exogenous DNA and membrane-disrupted sperm or demembranated sperm heads. CZB medium comprises 81.6 mM NaCl, 4.8 mM KCl, 1.7 mM CaCl₂, 1.2 mM MgSO₄, 1.8 mM KH₂ PO₄, 25.1 mM NaHCO₃, 0.1 mM Na₂ EDTA, 31 mM sodium lactate, 0.3 mM sodium pyruvate, 7 U/ml penicillin G, 5 U/ml streptomycin sulfate, and 4 mg/ml bovine serum albumin (BSA). The medium for oocyte collection from oviducts, subsequent treatments and micromanipulation was a modified CZB containing 20 mM Hepes, a reduced amount of NaHCO₃ (5 mM) and BSA (3 mg/ml). This medium is herein termed Hepes-CZB. For microinjection purposes, it was preferred to replace the BSA in the Hepes CZB with 0.1 mg/ml polyvinyl alcohol (PVA, cold water soluble, average molecular mass 10×10³) because PVA kept the wall of the injection pipette less sticky over a longer period of time than BSA and was beneficial during repeated use of a single pipette for multiple sperm head/oocyte transfers. The pH of both media was approximately 7.4. All oocyte manipulations were carried out in Hepes-buffered CZB (Hepes-CZB) under mineral oil at room temperature (23 degrees to 25 degrees Celsius) in air.

### Example 3

### Sperm Collection and Freezing

The caudae epididymes of a male were removed and punctured with sharply-pointed forceps. A drop of the dense mass of spermatozoa squeezed from the epididymes (about 2 μL), was placed in the bottom of a 1.5-ml polypropylene microcentrifuge tube (flat top) (Fisher Scientific, Pittsburgh, Pa.) and overlaid with 200 μL of the EGTA medium. The tube was incubated for 10 min at 37° C., to allow spermatozoa to disperse, and 30 μL aliquots were transferred to 1.5-ml polypropylene microcentrifuge tubes for freezing. The microcentrifuge tubes were plunged directly into liquid nitrogen (−196° C.) for 10 seconds, and then thawed by hand warming.

### Example 4

### Incubation with DTT

Immediately upon defrosting, the 30 μL aliquot was mixed with 10 μL of 4 mM dithiothreitol (DTT) in phosphate buffered saline. Concentrations of spermatozoa and DTT at this stage were ˜10⁶/mL and 1 mM, respectively. The mixture was then allowed to sit at 0° C. to 4° C. for 30 minutes. Control samples were not treated with DTT.

### Example 5

### Preparation of Exogenous Nucleic Acid

The enhanced green fluorescent protein (EGFP) transgene was a large (3.5 kb) Sal GI-Bam HI fragment of plasmid pCX-EGFP. The fragment harbors an EGFP gene expressed from a strong cytomegalovirus-IE-chicken β-actin enhancer-promoter combination, but lacks a eukaryotic origin of replication. (H. Niwa, K. Yamamura, J. Miyazaki, Efficient selection for high-expression transfectants with a novel eukaryotic vector, Gene 108, 193-199, 1991; G. Zhang, G. Vanessa, S. R. Kain, Biochem. Biophys. Res. Commun. 227, 707, 1996; T. Takada et al., Selective production of transgenic mice using green fluorescent protein as a marker, Nature Biotechnol., 15, 458-461, 1997). The 3.5 kb fragment containing the EGFP gene was obtained by digestion of the plasmid pCX-EGFP with the restriction enzymes Sal GI and Bam HI, and purified by methods known to those skilled in the art.

### Example 6

### Preparation of Mixtures of Exogenous Nucleic Acid and Spermatozoa

After standing for 30 minutes at 0° C. to 4° C., the linearized pCX-EGFP plasmid suspended in TE buffer was added such that the final DNA concentration in the medium was ˜6 ng/mL. One minute later, the spermatozoa-DNA mixture was mixed with 10% (w/v) polyvinyl pyrrolidone (PVP) in Hepes-buffered CZB medium such that the final PVP concentration was ˜7%. Since EGTA may interfere with normal activation of the oocyte (Izant, J. G., Chromosoma, 88:1-10, 1983; Groigno and Whitaker, Cell, 92:193-204, 1998), it was important to wash spermatozoa in another drop of the same medium containing 10% PVP, prior to ICSI, to minimize the introduction of EGTA into the oocyte cytoplasm.

### Example 7

### Preparation of Oocytes

Mature B6D2F1 (C57BL/6X DBA/2) female mice were induced to superovulate by consecutive injections of 7.5 International Units (IU) pregnant mare serum gonadotropin and 7.5 IU human chorionic gonadotropin (hCG) 48 hours apart. Fourteen hours after hCG injection, cumulus-oocyte complexes were collected from oviducts and treated with bovine testicular hyaluronidase (300 USP U/ml; ICN Biochemicals, Costa Mesa, Calif.) in Hepes-CZB medium for 3 minutes to disperse cumulus cells. Prior to injection with sperm nuclei, the oocytes were rinsed and stored in CZB medium under mineral oil equilibrated in 5% (v/v) CO₂ in air, at 37° C., for up to 4 hours

### Example 8

### Intracytoplasmic Sperm Injection

Intracytoplasmic sperm injection (ICSI) was carried out by modifying the technique originally described by Kimura and Yanagimachi (Biol. Reprod., 52:709-20, 1995), viz., the sperm injections were performed at room temperature (25° C.), rather than at 17° C. Further, only the sperm head was injected into the oocyte, instead of the entire spermatozoon. A single spermatozoon was drawn tail first into the injection pipette, and moved back and forth until the head-midpiece junction (the neck) was at the opening of the injection pipette. The head was then separated from the midpiece and tail by applying one or more Piezo pulses (Kimura and Yanagimachi, supra). After the midpiece and tail were discarded, the head was redrawn into the pipette and injected into an oocyte. The heads and tails of many freeze-dried spermatozoa were separated; therefore, their separation by Piezo pulses was unnecessary in most cases. ICSI was completed within 1 h of thawing of frozen spermatozoa, and within 2 hours of after sperm isolation from the epididymis. The amount of DNA-containing medium injected with the sperm head was ˜1 pL.

### Example 9

### Culture and Examination of Oocytes

Oocytes injected with a sperm head and exogenous DNA were incubated in CZB at 37° C. under mineral oil equilibrated in 5% (v/v) CO₂ in air and examined with an inverted microscope 5-6 hours later. Those with two distinct pronuclei and a second polar body were considered normally fertilized and cultured for 4 days in CZB.

Three to 3.5 days after micro-coinjection, embryos were examined for expression of GFP by epifluorescence microscopy with a UV light source (480 nm) with fluorescein isothiocyanate filters. This enabled the clear identification of nonfluorescent (non-GFP-expressing), weakly fluorescent, and strongly fluorescent embryos and mosaics, which were scored accordingly.

### Example 10

### Embryo Transfer

Normally fertilized oocytes reaching the morula or blastocyst stages were transferred into the uterine horns of recipient females (typically CD-1 albino females) that had been mated with vasectomized (CD-1) males three days previously to synchronize embryonic developmental stages with that of the uterine endometrium. A mean number of eight morulae/blastocysts were transferred into each horn. Females were allowed to deliver and raise their surrogate offspring. Some mature male and female offspring were randomly selected and mated to examine their fertility.

### Example 11

### Examination of Live Offspring for Transgene Expression

Live offspring obtained from embryos implanted in surrogate mothers, as described above, were examined one to 4 days after delivery for expression of ectopic GFP. EGFP expression was clearly observable as a green skin color under incidental illumination from a UV light source (480 nm).

### Example 12

### Analysis and Results of Data

Of 213 oocytes injected with DTT-treated sperm, 137 (64%) developed into morulae/blastocysts, 119 (87%) of which were EGFP positive. 25 (27%) of 94 transferred morulae/blastocysts developed into live offspring, 10 (40%) of which were EGFP positive. All of these 10 grew to become fertile adults, and their offspring were all EGFP positive.

In the control group, 135 (79%) of 170 ICSI oocytes developed into morulae/blastocysts, 103 (76%) of which were EGFP positive. 18 (33%) of 54 transferred morulae/blastocysts became live offspring, 2 (11%) of which were EGFP positive.

Accordingly, as shown below in Table 1, the use of EGTA and DTT increases the efficiency of ICSI-mediated transgenesis (defined herein as the proportion of transgenic offspring developed from 100 gene-injected eggs) to 6.8%, which represents a marked increase of efficiency in ICSI-mediated transgenesis performed without EGTA and DTT (1.8%, as described in U.S. Pat. No. 6,376,743, entitled “Mammalian transgenesis by intracytoplasmic sperm injection”, the entire contents of which are expressly incorporated by reference herein).

While the foregoing invention has been described in some detail for purposes of clarity and understanding, it will be appreciated by one skilled in the art, from a reading of the disclosure, that various changes in form and detail can be made without departing from the true scope of the invention in the appended claims.

TABLE 1
Comparison of the efficiency of ICSI-mediated transgenesis as assessed
by the proportion of live offspring produced by injection of transgene,
green-fluorescence protein (GFP) gene, into oocytes

| | No. gene | | No. | No. (%) | |
|---|---|---|---|---|---|
| | injected | No. (%) | transferred | transgenic | Overall |
| Method | oocytes | embryos | embryos | offspring | efficiency |
| No EGTA + | 313 | 155 (49.5) | 53 | 2 (3.7) | 1.8% |
| No DTT | | | | | |
| EGTA and | 213 | 137 (64.3) | 94 | 10 (10.6) | 6.8% |
| DTT | | | | | |

What is claimed is:

1. A method of preparing a spermatozoon suitable for use in ICSI-mediated transgenesis, wherein the method comprises the steps of:
(a) suspending the spermatozoon in a buffered medium, wherein the buffered medium comprises an ion-chelating agent;
(b) treating the spermatozoon to obtain a membrane-disrupted or demembranated spermatozoon; and
(c) incubating the membrane-disrupted or demembranated spermatozoon with an exogenous nucleic acid for a period of time.

2. The method of claim 1, wherein the spermatozoon is a sperm head.

3. The method of claim 1, wherein the buffered medium is a suspension medium.

4. The method of claim 3, wherein the suspension medium is CZB medium.

5. The method of claim 3, wherein the suspension medium is Tris-HCl buffer.

6. The method of claim 1, wherein the ion-chelating agent is a divalent-ion-chelating agent.

7. The method of claim 6, wherein the divalent-ion-chelating agent is a calcium-chelating agent.

8. The method of claim 7, wherein the calcium-chelating agent is EGTA.

9. The method of claim 6, wherein the concentration of the calcium-chelating agent is between 0.1 mM and about 200 mM.

10. The method of claim 9, wherein the concentration of the calcium-chelating agent is between 1 mM and about 100 mM.

11. The method of claim 10, wherein the concentration of the calcium-chelating agent is between 40 mM and about 60 mM.

12. The method of claim 11, wherein the concentration of the calcium-chelating agent is 50 mM, and the calcium-chelating agent is EGTA.

13. The method of claim 1, wherein the pH of the buffered medium is between 7.2 and 8.6.

14. The method of claim 13, wherein the pH of the buffered medium is between 7.4 and 8.2.

15. The method of claim 14, wherein the pH of the buffered medium is 8.2, the concentration of the ion-chelating agent in the buffered medium is 50 mM, and the ion-chelating agent is EGTA.

16. The method of claim 1, wherein the treatment comprises freezing and thawing the spermatozoon to obtain the membrane-disrupted spermatozoon.

17. The method of claim 1, wherein the treatment comprises freeze-drying and rehydrating the spermatozoon to obtain the membrane-disrupted spermatozoon.

18. The method of claim 1, wherein the treatment comprises extracting the spermatozoon with detergent to obtain the demembranated spermatozoon.

19. The method of claim 1, wherein the exogenous nucleic acid comprises more than one transgene.

20. The method of claim 1, wherein the exogenous nucleic acid comprises a cDNA.

21. The method of claim 1, wherein the exogenous nucleic acid comprises genomic DNA.

22. The method of claim 1, wherein the incubating time period with the exogenous nucleic acid is sufficient to form a close association between the exogenous nucleic acid and the membrane-disrupted or demembranated spermatozoon.

23. The method of claim 22, wherein the incubating time period if about 30 seconds to 5 minutes.

24. The method of claim 23, wherein the incubating time period with the exogenous nucleic acid is about 45 seconds to 3 minutes.

25. The method of claim 24, wherein the incubating time period with the exogenous nucleic acid is about 1 minute to 2 minutes.

26. The method of claim 25, wherein the incubating time period with the exogenous nucleic acid is about 1 minute.

27. The membrane-disrupted or demembranated spermatozoon of claim 1, wherein the exogenous nucleic acid is closely associated with the membrane-disrupted or demembranated spermatozoon.

28. The method of claim 1, further comprising the step of incubating the membrane-disrupted or demembranated spermatozoon with a disulfide reducing agent for a period of time, followed by incubation of the membrane-disrupted or demembranated spermatozoon with the exogenous nucleic acid.

29. The method of claim 28, wherein the disulfide reducing agent is contained in a buffered medium.

30. The method of claim 29, wherein the buffered medium is a suspension medium.

31. The method of claim 30, wherein the buffered medium is a buffered saline solution.

32. The method of claim 28, wherein the disulfide reducing agent is one of DTT, TCEP, TCP, TRX or GSH.

33. The method of claim 32, wherein the disulfide reducing agent is DTT.

34. The method of claim 33, wherein the concentration of DTT during incubation with the membrane-disrupted or demembranated spermatozoon is between 0.1 mM and about 50 mM.

35. The method of claim 34, wherein the concentration of DTT during incubation with the membrane-disrupted or demembranated spermatozoon is between 1 mM and about 5 mM.

36. The method of claim 35, wherein the concentration of DTT during incubation with the membrane-disrupted or demembranated spermatozoon is 1 mM.

37. The method of claim 28, where the incubation time period of the membrane-disrupted or demembranated spermatozoon with the disulfide reducing agent is sufficient to result in partial or complete nuclear decondensation of the membrane-disrupted or demembranated spermatozoon.

38. The method of claim 37, wherein the incubation time period is about 5 minutes to 1 hour.

39. The method of claim 38, where the incubation time period is about 10 minutes to 50 minutes.

40. The method of claim 39, where the incubation time period is about 20 minutes to 40 minutes.

41. The method of claim 40, where the incubation time period is about 30 minutes, and the disulfide reducing agent is DTT in 1 mM concentration.

42. The membrane-disrupted or demembranated spermatozoon of claim 28, wherein the exogenous nucleic acid is closely associated with the membrane-disrupted or demembranated spermatozoon.

43. A method for obtaining a transgenic embryo, comprising the steps of:
(a) coinserting the spermatozoon of claim 1 and the exogenous nucleic acid into an unfertilized oocyte to form a transgenic fertilized oocyte; and
(b) allowing the transgenic fertilized oocyte to develop into a transgenic embryo.

44. The method of claim 43, further comprising the step of transplanting the transgenic embryo into a surrogate mother and allowing the transgenic embryo to develop into a live offspring.

45. A method for obtaining a transgenic embryo, comprising the steps of:
(a) coinserting the spermatozoon of claim 28 and the exogenous nucleic acid into an unfertilized oocyte to form a transgenic fertilized oocyte; and
(b) allowing the transgenic fertilized oocyte to develop into a transgenic embryo.

46. The method of claim 45, further comprising the step of transplanting the transgenic embryo into a surrogate mother and allowing the transgenic embryo to develop into a live offspring.
